(19) United States
(12) Patent Application Publication
(10) Pub. No.: US 2002/0151919 A1
(43) Pub. Date: Oct. 17, 2002

(54) Roller cutting device, cutting template and method for cutting tissue

(76) Inventors: Jack W. Love, Santa Barbara, CA (US); Robert W. Suggitt, Burnaby (CA)

Correspondence Address: Christopher J. Hayes, Bryan Cave LLP, 211 North Broadway, Suite 3600, St. Louis, MO, 63102-2750, US

(21) Appl. No.: 10/121,420

(22) Filed: Apr. 12, 2002

Related U.S. Application Data
(60) Provisional application No. 60/283,401, filed on Apr. 12, 2001.

(51) Int. Cl. A61B 17/32

(52) U.S. Cl. 606/180

(57) ABSTRACT

The present invention relates to an innovative roller cutting device, cutting template and method for cutting a tissue pattern. The invention allows a precise tissue pattern to be cut without dragging the material and possibly tearing the tissue as would often occur using a straight scalpel blade. The roller cutter and template described herein can be used for numerous surgical applications requiring precise cutting of tissue and other materials.

### CROSS-REFERENCE TO RELATED APPLICATION

This application claims the benefit of U.S. Provisional Application No. 60/283,401, filed Apr. 12, 2001.

### FIELD OF THE INVENTION

The present invention pertains to the field of surgical instruments. More particularly, this invention relates to a roller cutting device, cutting template and a method for cutting tissue.

### BACKGROUND OF THE INVENTION

To overcome disadvantages of the prior methods of valve reconstruction, Cardiomend LLC (the assignee of the present application), developed improved, more easily reproducible, less complicated, and generally standardized, methods of reconstructing heart valves. These methods are generally described in Love U.S. Pat. Nos. 5,716,399, Love 6,129,758, and copending U.S. application Ser. No. 09/330,689 (the “Love patents”), each of which is incorporated herein by reference in its entirety. The Love patents generally describe a method for reconstructing semilunar valves using a novel unitary trefoil tissue pattern preferably of lightly tanned autologous pericardium. This method preferably includes the steps of (a) sizing of the heart valve based on the distance between adjacent commissures of the native valve annulus; (b) cutting a trefoil tissue pattern corresponding in size to the native valve annulus; (c) temporarily mounting the tissue pattern on a surgical instrument for holding the tissue in a configuration of a circulatory system valve to facilitate attachment of the tissue; and (d) suturing the tissue to the native valve annulus.

The present invention relates to an innovative roller cutting device, cutting template and cutting method particularly adapted for cutting a tissue pattern for use in the method described in the Love patents. Among the advantages of the present invention is that it allows a precise tissue pattern to be cut without dragging the material and possibly tearing the tissue as would often occur using a straight scalpel blade. The roller cutter and template described herein can also be used for numerous other surgical applications requiring precise cutting of tissue and other materials.

### SUMMARY OF THE INVENTION

The invention includes a tissue cutting device for use with a scalpel handle. The scalpel handle is preferably a conventional handle having a ridge on which a blade can be mounted. The cutting device includes a rotary blade rotatively attached to a blade holder. The blade holder has slot therein. The slot allows the holder to be attached to the scalpel handle by inserting the ridge of the handle into the slot of the holder.

The invention also includes a template for cutting a piece of tissue into a desired pattern. The template has a base and a cover. The base has one or more channels therein corresponding in shape to the desired pattern. The piece of tissue can be placed between the base and the cover and cut into the desired pattern using the channels as a guide.

In one embodiment, the one or more channels comprise an inner channel and an outer channel sized to correspond to the geometry of a heart valve. The outer channel conforms to the size and shape of the valve annulus and the inner channel conforms to the size and shape of the valve orifice.

A kit can be provided for cutting a piece of tissue into a desired pattern. The kit includes a tissue cutting device having a rotary blade and a template. The template has a base and a cover. The cover has one or more channels therein corresponding in shape to the desired pattern. The piece of tissue is placed between the base and the cover and cut into the desired pattern by guiding the rotary blade through the channels. The rotary blade is preferably sized to fit within the channels such that the lateral movement of the blade within the channels is limited to provide an even, accurate cut.

### DRAWINGS

These, and other features, aspects and advantages of the present invention will become more fully apparent from the following detailed description, and accompanying drawings where:

FIG. 1 is a side perspective view of a roller cutter;

FIG. 2A is a side view of a roller cutter;

FIG. 2B is a front sectional view of the rivet and blade of a roller cutter taken along line A-A in FIG. 2A;

FIG. 3A is a side view of a roller cutter;

FIG. 3B is a sectional view of the rivet and blade of a roller cutter taken along line A-A in FIG. 3A;

FIG. 4 is a side view of a blade holder of a roller cutter;

FIG. 5A is a side view of a rotary blade of a roller cutter;

FIG. 5B is a front view of a rotary blade of a roller cutter;

FIG. 6A is a side view of a rivet of a roller cutter;

FIG. 6B is a front view of a rivet of a roller cutter;

FIG. 6C is a front sectional view of a rivet of a roller cutter taken along line B-B in FIG. 6A;

FIG. 7A is a side view of a roller cutter and a standard scalpel blade handle for use therewith;

FIG. 7B is a prospective view of a roller cutter with attached scalpel handle used with a cutting template;

FIG. 8A is a front view of an open cutting template for cutting a trefoil tissue pattern for semilunar valve reconstruction;

FIG. 8B is a front perspective view of an open cutting template for cutting a trefoil tissue pattern for semilunar valve reconstruction; and

FIG. 8C is a back perspective view of an open cutting template for cutting a trefoil tissue pattern for semilunar valve reconstruction.

### DETAILED DESCRIPTION OF THE INVENTION

The subject invention provides new, useful and unique devices and method for cutting tissue. The instruments and method are particularly suited for cutting tissue for reconstruction of cardiac semilunar valves (aortic and pulmonic) utilizing the methods described in Love patents incorporated herein by reference.

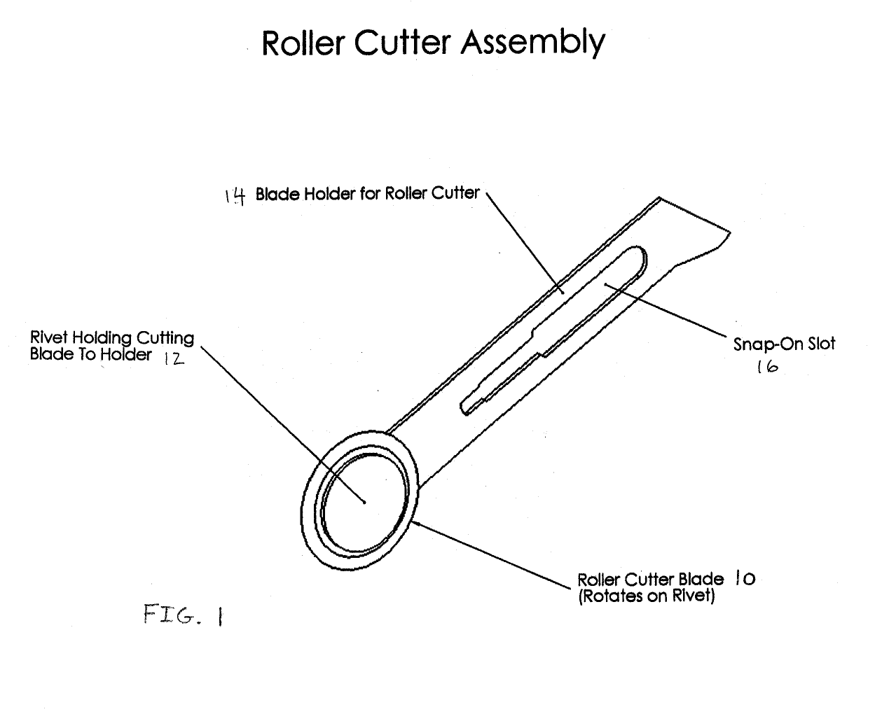

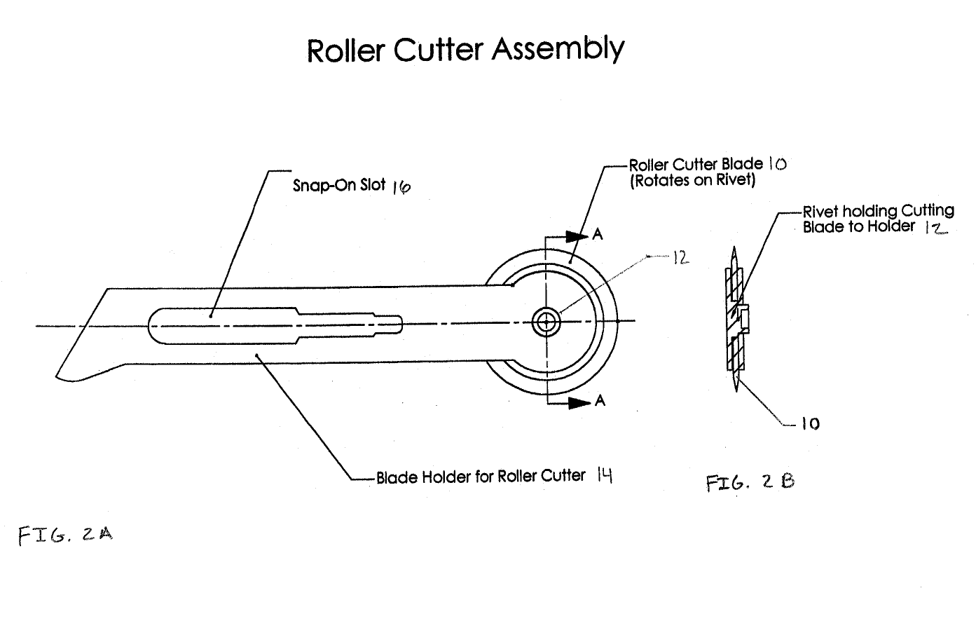

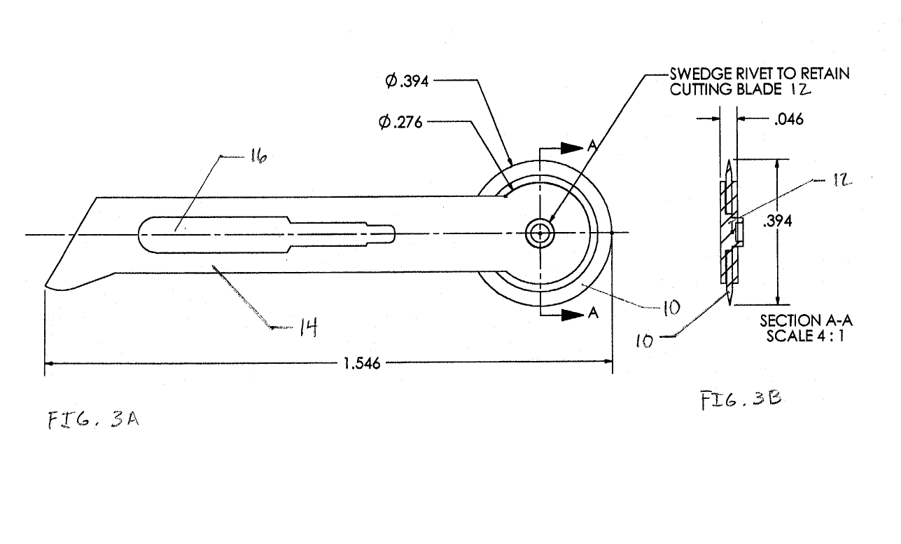

As shown in FIGS. 1, 2A, 2B, 3A, and 3B, the roller cutter comprises the following principal parts: a rotary blade 10, a blade holder 14 for holding the blade 10; and a rivet 12 for attaching the blade 12 to the holder 14 such that blade 10 can rotate about rivet 12. All parts are preferably constructed of stainless steel so that the device can be readily sterilized and discarded after single use.

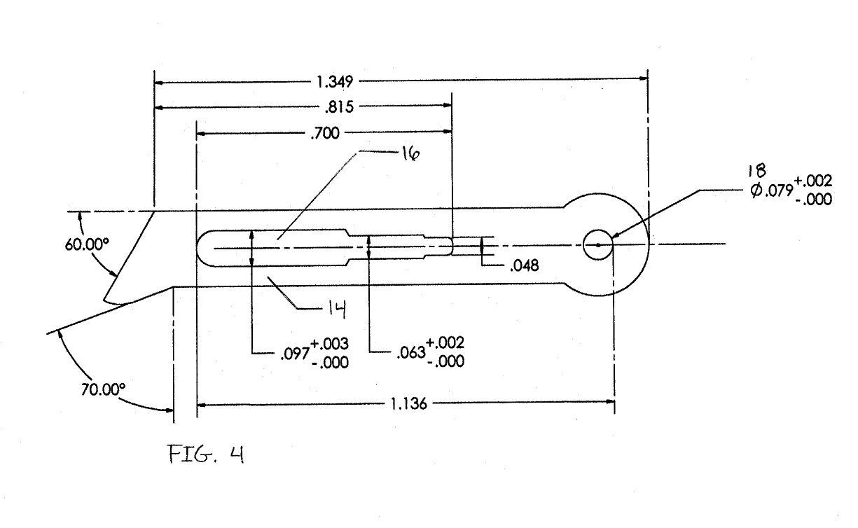

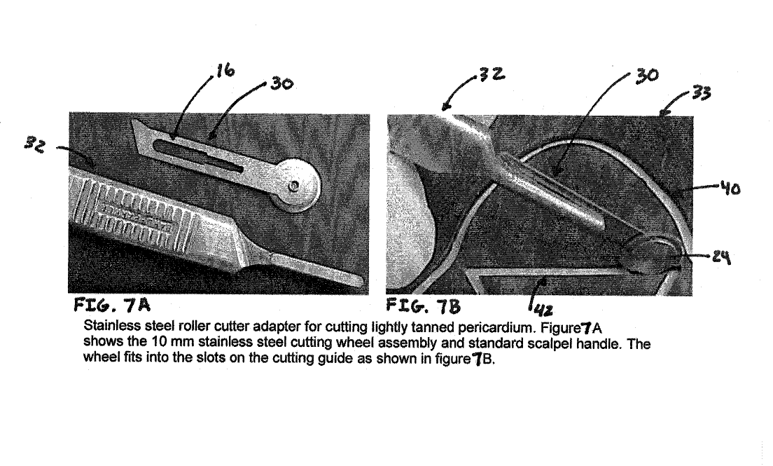

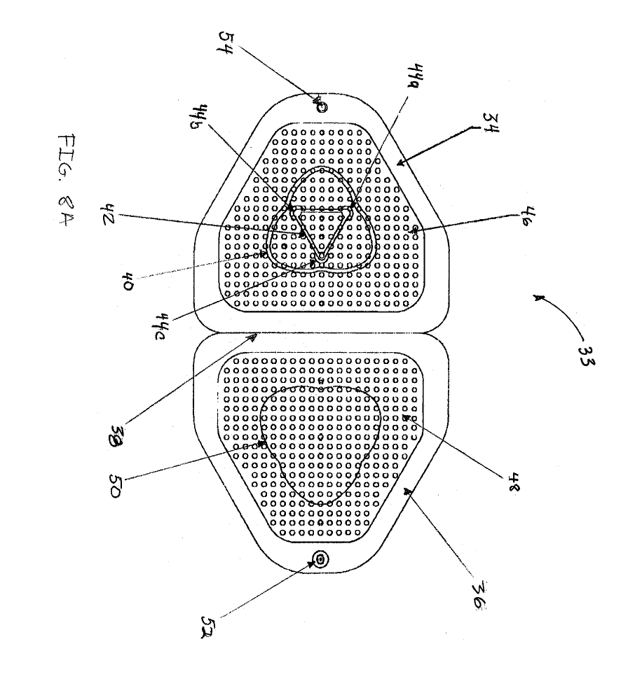

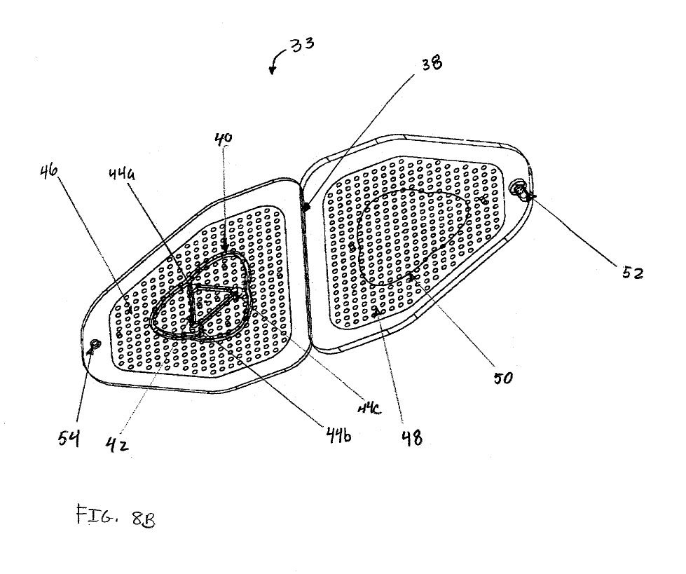

An embodiment of blade holder 14 is illustrated in FIG. 4. Holder 14 comprises a slot 16, which is preferably sized to allow holder 14 to be attached to a standard scalpel handle as illustrated in FIGS. 7A and 7B. Blade holder 14 also comprises a circular opening 18 for receiving rivet 12. The blade holder 14 is preferably constructed from an approximately 0.015 inch sheet of 440A stainless steel.

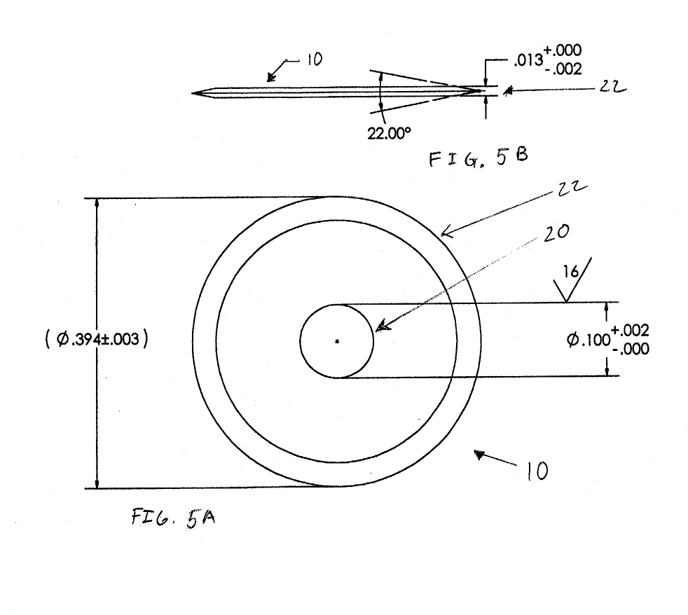

An embodiment of rotary blade 10 is illustrated in FIGS. 5A and 5B. Blade 10 is circular and preferably includes a circular center opening 20 for receiving rivet 12. Blade 10 also includes an outer center sharpened rim 22, which is preferably cut or ground to an angle of approximately 22 degrees from each side surface of blade 10. Blade 10 is preferably has a diameter of about 0.394 inch and is constructed of 440A stainless steel.

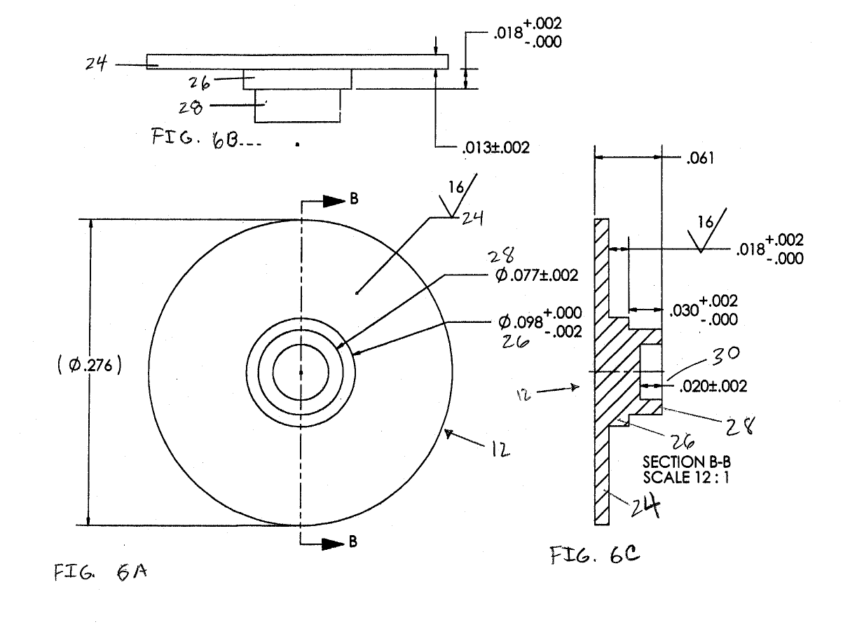

An embodiment of rivet 12 is illustrated in FIGS. 6A, 6B, and 6C. Rivet 12 preferably comprises cylindrical blade hub 26 which fits inside opening 20 of blade 10. Rivet 12 also includes cylindrical outer hub 28 which fits inside opening 18 of blade holder 14 to allow blade 10 to be rotatively mounted to holder 14. Outer hub 28 preferably includes a recessed area 30 for receiving a retainer for securing rivet 12 to holder 14. Rivet 12 further includes disk 24 for securing the rotary blade 10 on blade hub 26 such that blade 10 can rotates about rivet 12 at blade hub 26.

The roller cutter is designed to be used to cut precise geometric patterns used, for example, for heart valve reconstruction. As shown in FIG. 7A, the roller cutter 30 can preferably be used with a standard scalpel handle 32 by attaching holder 14 to the scalpel handle 32 using snap-on slot 16. As shown in FIG. 7B, the roller cutter 30 is used in a preferred embodiment to cut tissue into a precise geometric pattern for use in semilunar heart valve reconstruction using a cutting template 33.

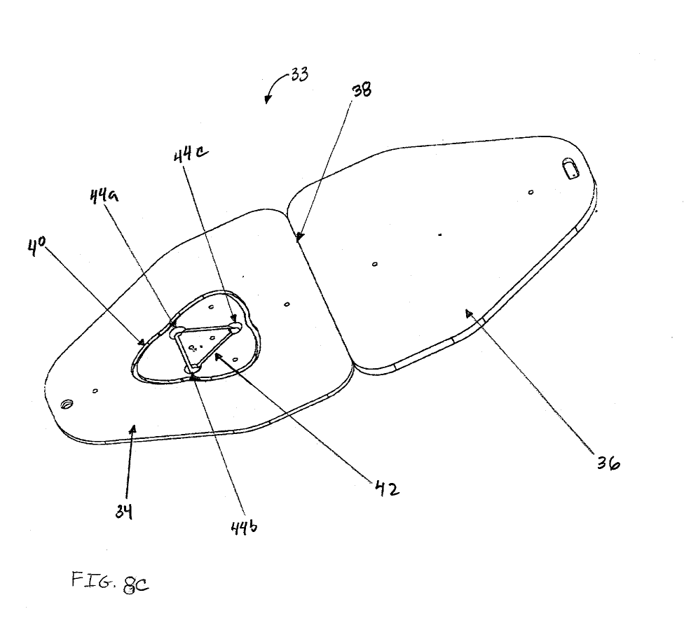

FIGS. 8A, 8B and 8C show an embodiment of a cutting template 33 for use with the roller cutter 30 for cutting a trefoil tissue pattern for semilunar heart valve reconstruction as is fully described in the Love patents. The template 33 preferably comprises a cover 34 and a base 36, connected by a hinge 38. Cover 34 has an outer channel 40 and an inner channel 42 forming the shape of the preferred tissue pattern. Channels 40 and 42 are preferably sized to correspond to the expected geometry of a native heart valve based upon measurements taken from the native valve annulus, and is preferably provided in various sizes to correspond to a range of typical annulus sizes (e.g., 6 templates can be provided corresponding to valve diameters of 19-29 mm in 2 mm increments). Outer channel 40 conforms to the expected size and shape of the valve annulus and inner channel 42 conforms to the expected size shape of the valve orifice. Inner channel 42 preferably includes rounded corners 44a, 44b and 44c in which a standard dermal biopsy punch can be inserted to facilitate cutting of the corner areas of the tissue pattern, which may be difficult to reach using the circular blade 10 of the roller cutter. Cover 34 also preferably includes a textured cover label 46, which is adhered to the inside surface of cover 34 to hold together the portions of cover 34 that are separated by channels 40 and 42. Cover label 46 is preferably constructed using biocompatible paper or plastic materials that can readily be cut through and includes a texturing (e.g., dimpling) to help hold the tissue in place. Base 36 also preferably includes a textured area 48 to help hold the tissue in place. Base 36 also preferably includes a pattern outline 50 of the trefoil tissue pattern mirroring outer channel 40 to provide a visual indication on base 36 of the minimum size of tissue needed to form the desired pattern. A locking mechanism 52 (e.g., a clip) on base 36 preferably cooperates with a locking mechanism 54 (e.g., a small hole) on cover 34 to secure the cover 34 and base 36 in the closed position.

The template 33 is used by opening it and placing a piece of tissue on the base 36 that is large enough to cover at least pattern outline 50. The cover 34 is then closed and secured against base 36 with the tissue piece between the cover 34 and the base 36. As shown in FIG. 7B, with the template locked in the closed position with tissue between cover 34 and base 36, blade 10 of roller cutter 30 is inserted into and cuts the tissue along channels 40 and 42. Blade 10 and disk 24 of rivet 12 are preferably sized to fit into channels 40 and 42 of template cover 34 such that disk 24 acts as a guide limiting the lateral movement of blade 10 within channels 40 and 42 to provide an even, accurate cut. Disk 24 also serves to prevent blade 10 from cutting the edges of template surrounding channels 40 and 42 to avoid possible contamination of the tissue resulting from shaved template particles. The corners of the valve orifice are preferably cut by inserting a standard dermal biopsy punch into corners 44a, 44b, and 44c. After cutting, the trefoil tissue pattern is preferably removed from the template 33 and placed on a tissue holding instrument to facilitate surgical attachment of the tissue to the native valve annulus as described in the Love patents.

The many advantages of devices and method described herein include the ability to cut slippery material (such as pericardium tissue) without the movement of material typically experienced when using conventional scalpel blades. The roller cutter cuts with a shearing action. By avoiding dragging movement associated with conventional blades, the roller cutter reduces the risk of uneven cutting of the tissue as a result of blade friction. The roller cutter cuts an extremely clean edge. The roller cutter can be used with standard scalpel handles and can be cost-effectively constructed of materials allowing disposal after a single use.

Although the invention has been described with reference to specific embodiments, it will be understood by those skilled in the art that various changes may be made without departing from the spirit or scope of the invention. For instance, the numerous details set forth herein (e.g., details relating to the presently preferred embodiment of the device for use in connection with semilunar valve reconstruction) are provided to facilitate the understanding of the invention and are not provided to limit the scope of the invention. Those skilled in the art will appreciate that the devices and method described herein are readily adaptable for example for any application requiring the cutting of precise geometric patterns from tissue or other materials. Accordingly, the disclosure of embodiments of the invention is intended to be illustrative of the scope of the invention and is not intended to be limiting.

We claim:

1. A tissue cutting device for use with a scalpel handle, the device comprising:
a. a blade holder having a slot for allowing the holder to be attached to the scalpel handle; and
b. a rotary blade attached to the holder.

2. The tissue cutting device of claim 1, wherein the rotary blade is a circular blade rotatively attached to the holder.

3. A template for cutting a piece of tissue into a desired pattern, the template comprising:
a. a base; and
b. a cover having one or more channels therein corresponding in shape to the desired pattern,
whereby the piece of tissue can be placed between the base and the cover and cut into the desired pattern using the channels as a guide.

4. The template of claim 3, wherein the one or more channels comprise an inner channel and an outer channel sized to correspond to the geometry of a heart valve having a valve annulus and a valve orifice, wherein the outer channel conforms to the size and shape of the valve annulus and the inner channel conforms to the size and shape of the valve orifice.

5. A kit for cutting a piece of tissue into a desired pattern, the kit comprising:
a. a tissue cutting device having a rotary blade; and
b. a template for cutting a piece of tissue into a desired pattern, the template comprising:
1. a base; and
2. a cover having one or more channels therein corresponding in shape to the desired pattern,
whereby the piece of tissue can be placed between the base and the cover and cut into the desired pattern by guiding the rotary blade through the channels.

6. The kit of claim 5, wherein the rotary blade is sized to fit within the channels such that the lateral movement of the blade within the channels is limited.

7. A method for cutting a piece of tissue into a desired pattern, the method comprising:
a. providing a tissue cutting device having a rotary blade;
b. providing a template comprising:
1. a base; and
2. a cover having one or more channels therein corresponding in shape to the desired pattern;
c. placing the piece of tissue between the base and the cover of the template; and
d. cutting the piece of tissue into the desired pattern by guiding the rotary blade through the channels.
